(19) United States
(12) Patent Application Publication
(10) Pub. No.: US 2011/0184361 A1
(43) Pub. Date: Jul. 28, 2011

(54) Connecting device for use with vacuum treatment of wounds

(76) Inventors: Pierre Crojzat, Herbrechtingen (DE); Axel Eckstein, Heidenheim (DE); Juergen Hofstetter, Heidenheim (DE)

(21) Appl. No.: 12/893,023

(22) Filed: Sep. 29, 2010

(30) Foreign Application Priority Data
Jan. 26, 2010 (DE) 10 2010 006 273.1

(51) Int. Cl. A61M 1/00

(52) U.S. Cl. 604/319

(57) ABSTRACT

A connecting device (2) for use for the treatment of wounds with vacuum, comprises a conduit means (4) which can be loaded with vacuum, and an extensive vacuum-tight first carrier means (6) for the conduit means (4) on which the conduit means (4) is held in a vacuum-tight fashion, wherein the carrier means (6) can be disposed onto a vacuum dressing which extends over the wound and tightly seals it from the atmosphere. The conduit means (4) communicates with the wound space through at least one opening (14) in the wall of the conduit means (4) facing the vacuum dressing and through at least one opening in the vacuum dressing. The connecting device is improved in that the conduit means (4) is flexible and flat and a broad recess (10) is formed in the first carrier means (6), the width of which is at least as large as the width of the conduit means (4) and the length of which is at least 15 mm, and the conduit means (4) flatly spreads out into this recess (10) and thereby directly faces the vacuum dressing via this recess (10) during use, such that the at least one opening (14) in the conduit means (4) directly communicates with the at least one opening in the vacuum dressing.

The invention concerns a connecting device for use with vacuum treatment of wounds, comprising a conduit means which can be evacuated, and a flat vacuum-tight first carrier means for the conduit means, on which the conduit means is held in a vacuum-tight fashion, wherein the carrier means can be disposed onto a vacuum dressing which extends over the wound and tightly seals it from the atmosphere, wherein the conduit means communicates with the wound space through at least one opening in the wall of the conduit means facing the vacuum dressing and through at least one opening in the vacuum dressing.

The treatment of wounds with vacuum, in particular deep wounds the healing process of which is a priori problematic, has recently become more and more important. Vacuum treatment thereby means that an area of a body or wound, which is, in principle, exposed to the surrounding atmosphere, is sealed from the surroundings, i.e. the atmosphere in which we live and breathe, in a pressure-tight or vacuum-tight fashion through means which are described in detail below, wherein a lower pressure than atmospheric pressure, consequently a vacuum compared to atmosphere, can be created and permanently maintained within the sealed wound area in a fashion which is also explained below. The vacuum mentioned in this connection means a pressure range which is typically between 0 and 500 mg Hg (mm mercury column) below the ambient atmospheric pressure. This has proven to support wound healing. A vacuum dressing is provided for vacuum-tight sealing, which may comprise e.g. a pressure-tight or vacuum-tight foil layer which is typically applied to a sound body area surrounding the wound to thereby tightly seal it. For introducing and maintaining a vacuum in the wound space on the basis of a means that generates vacuum, i.e. a vacuum pump in the broadest sense, the present systems for vacuum therapy of wounds may utilize conduit means which can be loaded with a vacuum and cooperate with the vacuum dressing by means of a connecting device in order to supply vacuum to or into the wound space. A connecting device of the above-mentioned type is disclosed e.g. in WO 2006/052338 A2. It utilizes tubular hoses for transporting the vacuum to the wound.

It is the underlying purpose of the present invention to create a connecting device of the above-mentioned type which is comfortable for the patient or causes minimum pain upon load or contact, and the components of which can be sealed with technically and economically acceptable expense in a user-friendly fashion.

This object is achieved in accordance with the invention in a connecting device of the above-mentioned type in that the conduit means is designed to be flexible and flat and a broad recess is formed in the first carrier means, the width of which is at least as large as the width of the conduit means and the length of which is at least 15 mm, and the conduit means widely extends into this recess and thereby directly faces the vacuum dressing through this recess during use, such that the at least one opening in the conduit means directly communicates with the at least one opening in the vacuum dressing.

The conduit means therefore does not have a tubular shape with a substantially round cross section but a flat shape, wherein the extension in width is substantially larger than its thickness. In combination with the material selected, this yields a flexible conduit means which is more comfortable for the patient when contact pressure is applied to the connecting device or the conduit means. Point loading is thereby also reduced, which otherwise would cause pain and is highly problematic, in particular, in case of fresh wounds which are sensitive to pain. The flat design also reduces the danger of becoming caught or jammed. Due to the flat design of the conduit means and the carrier means, the pressure load is distributed onto a larger surface area of the wound dressing, which is very advantageous in view of the above-mentioned problems.

Since, in accordance with the invention, a broad recess is formed in the first carrier means, into which a longitudinal section of the flat conduit means widely spreads and thereby faces the vacuum dressing through this recess, preferably without interposing further materials, or abuts the vacuum dressing, the extension in thickness in this sensitive area of the connecting device is further reduced, which is advantageous in view of the above-described problems.

The broad recess in the carrier means could indeed basically have an open edge such that the conduit means can continuously extend from the outside into this recess. Due to the sealing problems that arise in this connection, one embodiment is preferred in which the broad opening in the carrier means is surrounded in the peripheral direction. The conduit means then initially extends such that it extensively abuts the equally extensive carrier means in order to then further extend into the broad recess in the carrier means. This facilitates sealing with respect to the carrier means and atmosphere, which is described in detail below.

In a further design of the inventive idea, the length of the recess is advantageously at least 20 mm, in particular at least 30 mm and moreover in particular at least 40 mm.

The inventive connecting device can be disposed on or adhered to the upper side of the vacuum dressing facing away from the wound in any feasible fashion, i.e. by means of a bonding agent or by additional tape-like or foil-like means. In one preferred embodiment of the invention, the first carrier means has on its side facing the vacuum dressing an adhesive coating or is designed to be self-adhesive in this area, such that the connecting device can be directly stuck onto an upper side of the vacuum dressing facing away from the wound and be applied in a vacuum-tight fashion. In accordance with this embodiment, the carrier means and its adhesive coating realize the preferably detachable adhesive bond with the vacuum dressing.

In one further design of the invention, the conduit means is advantageously held in a vacuum-tight fashion on the first carrier means in that a second extensive carrier means extends over it such that it is received like a sandwich between the first and the second carrier means, wherein the second carrier means completely extends over the recess in the projection on the plane of the first carrier means. It has turned out that, in accordance with this further inventive idea, the conduit means can be economically produced and sealed with respect to the carrier means in a compact fashion. In accordance with this inventive idea, flatly overlapping, i.e. flat, mutually abutting flexible sheet materials are provided which can be well sealed with respect to each other due to their extensive abutment.

It is thereby advantageous for the second carrier means to be offset with respect to the extension of the first carrier means in the area of the outlet of the conduit means such that a non-overlapping area of the first conduit means is formed at that location.

For sealing, in particular, in the outlet area of the conduit means between the carrier means, the materials could be joined directly, in particular thermally like a vulcanization bond, or a sealing material, in particular on the basis of silicon, could be used for sealing. It may be sufficient to provide the sealing material only in the outlet area of the conduit means between the carrier means, thereby sealing the area around the conduit means. The sealing material may simultaneously act like a bonding agent between the first and the second carrier means.

It is moreover advantageous for the first and/or the second carrier means to be formed from or contain a thermoplastic elastomer, in particular of silicon or polyurethane or preferably of a hydrocolloid material, in particular a self-adhesive hydrocolloid material. A hydrocolloid material is thereby preferred. A hydrocolloid material is defined as a matrix mass which contains homogeneously dispersed hydrocolloids (e.g. sodium carboxymethyl cellulose). A self-adhesive hydrocolloid material is defined as a pressure-sensitive adhesive (e.g. on the basis of a synthetic rubber) which contains homogeneously dispersed hydrocolloids (e.g. sodium carboxymethyl cellulose).

Hydrocolloid sheet materials which are suited for use as wound support are available from the company Avery Dennison (Turnhout, Belgium) under the trade name MED 5598H. The extensive carrier means, in particular with or consisting of hydrocolloid material, may comprise a carrier film, e.g. a polyurethane film and/or a detachable release coating. One side of the above-mentioned hydrocolloid sheet materials comprises a polyurethane film and opposite thereto a detachable release coating.

The elastomeric material from which the conduit means is formed preferably has a Shore A-hardness of maximally 60, in particular 5 to 60, in particular 10 to 60, in particular 15 to 50, in particular 15 to 40 and moreover in particular 15 to 35. The Shore-A hardness is determined in accordance with DIN 53505 of August 2000, i.e. at 23° C. on a plate-shaped flat and smooth sample body of a thickness of 6 mm as described in the standard. In accordance with a preferred embodiment of the invention, the conduit means is formed on the basis of silicon.

As mentioned above, the inventive connecting device is very flat in the direction perpendicular to the plane of the vacuum dressing. This extension in thickness of the composite of conduit means and carrier means is preferably maximally 10 mm, in particular maximally 7 mm, in particular maximally 6 mm and moreover in particular maximally 5 mm.

The width of the flat conduit means is at least 10 mm, in particular at least 15 mm and moreover, in particular at least 18 mm and, in particular maximally 30 mm and moreover, in particular maximally 25 mm.

Since the flat and flexible conduit means is used to supply vacuum into the wound space and, if necessary, to supply rinsing liquids or rinsing gases and to discharge wound secretion, i.e. it preferably only has a channel-forming communication function, it is proposed not to form the conduit means in a laminated fashion with several components or layers but to form it, despite its flat design, in a tubular shape, i.e. having a cross-section extending continuously in one piece from one single material in the peripheral direction.

It is also advantageous for the conduit means to have means which are formed on the interior and are designed, in particular, in one piece with the material of the conduit means for preventing collapse of the conduit means in case of vacuum load. These means for preventing collapse of the conduit means may, in particular, be provided in a conduit means of tubular shape as described above. These means for preventing collapse may e.g. be formed by ribs or projections. In a further development of this inventive idea, they advantageously extend in a continuous fashion. The conduit means can then advantageously be formed as an extrusion element.

It may moreover be advantageous for the conduit means to comprise several channels which are separated from each other in a pressure-tight fashion, wherein, in this case, the conduit means is also preferably formed in one piece, i.e. does not comprise a combination of several separate channel-forming means. The channels may comprise a rinsing channel which may guide a rising medium towards the end of the connecting device facing the wound, and a channel carrying a vacuum which is used to supply vacuum or is used to discharge wound secretions. Any clogging within the conduit means may thereby also be eliminated. Each channel thereby communicates with at least one opening in the conduit means.

The flat conduit means preferably extends over a certain distance in the longitudinal direction and may then merge into a normal, more torsion-resistant round hose via a transition or coupling element (not shown) which may form a plug connection or adhesive connection. The round hose is guided to a device which generates vacuum and may be designed in the form of a stationary device or a portable device that can be carried on the body of the patient. The transition or coupling element may also be designed for coupling a multi-channel conduit means with a multi-channel round hose. A section of 10 to 60 cm has turned out to be a useful longitudinal extension of the flat conduit means.

The extensive first and/or second carrier means of the connecting device with which the flat conduit means is connected in a vacuum-tight fashion by the manufacturer is/are preferably also formed from a flexible elastomeric material of a Shore A-hardness of 5 to 60, in particular of 10 to 60, in particular of 15 to 50, in particular of 15 to 40 and moreover, in particular of 15 to 35. The flat carrier means advantageously has a thickness of 0.75 to 3 mm, in particular of 1 to 3 mm.

The carrier means is used to hold and uniformly support the flat conduit means. Its breadth is therefore larger than the breadth of the conduit means in the relevant longitudinal end on the side of the wound. It is thereby advantageous for the flat extension of the first carrier means to be at least 1.5 times, preferably at least two times the area of the conduit means projected perpendicularly onto the carrier means, since the forces introduced via the conduit means upon contact are thereby distributed over a larger area and moreover bending moments that act on the conduit means are not or only minimally transferred to the vacuum dressing. Acceptance thereof is facilitated by the plate-shaped carrier means. It has turned out to be sufficient for the above-mentioned area ratio to be at most 5, in particular at most 4, wherein a ratio of 2 to 3 has proven to be advantageous.

The flat conduit means could e.g. have a rectangular shape as viewed in cross-section, wherein the two narrow sides could also preferably be rounded. In accordance with a further embodiment of the invention, the conduit means has a trapezoidal shape as viewed in cross-section. The narrow sides then drop e.g. at an inclined angle relative to the plane of the flat carrier means of 25° to 60°, in particular 35° to 50°, wherein the flanks of the single-sided or preferably two-sided trapezoidal shape do not necessarily have to extend in a straight line but may also be rounded.

In view of the number and size of the openings in the flat side facing the vacuum dressing, or in the wall of the conduit means, it would, in principle, be feasible to only provide one single opening which communicates with a corresponding opening in the vacuum dressing and thereby with the wound space. However, it has turned out to be advantageous to provide several openings, in particular at least two openings per each cm of length of the conduit means in the area of the longitudinal section of the conduit means facing the vacuum dressing. It is thereby also advantageous for the clearance area of the openings in the conduit means to be between 5 and 50% of the area of the recess in the carrier means.

The side of the connecting device facing the vacuum dressing and being provided with an adhesive coating is advantageously entirely provided with a removable release layer even in the area of the recess, which is directly released prior to application of the connecting device onto the vacuum dressing.

Each of the above-described features is regarded as being important to the invention by itself and in arbitrary combination with each other and with further features. Further features, details and advantages of the invention can be extracted from the following claims and the drawing and following description of a preferred embodiment of the invention.

In the drawing:

FIG. 1 shows a perspective view of an inventive connecting device for use for the treatment of wounds with a vacuum dressing;

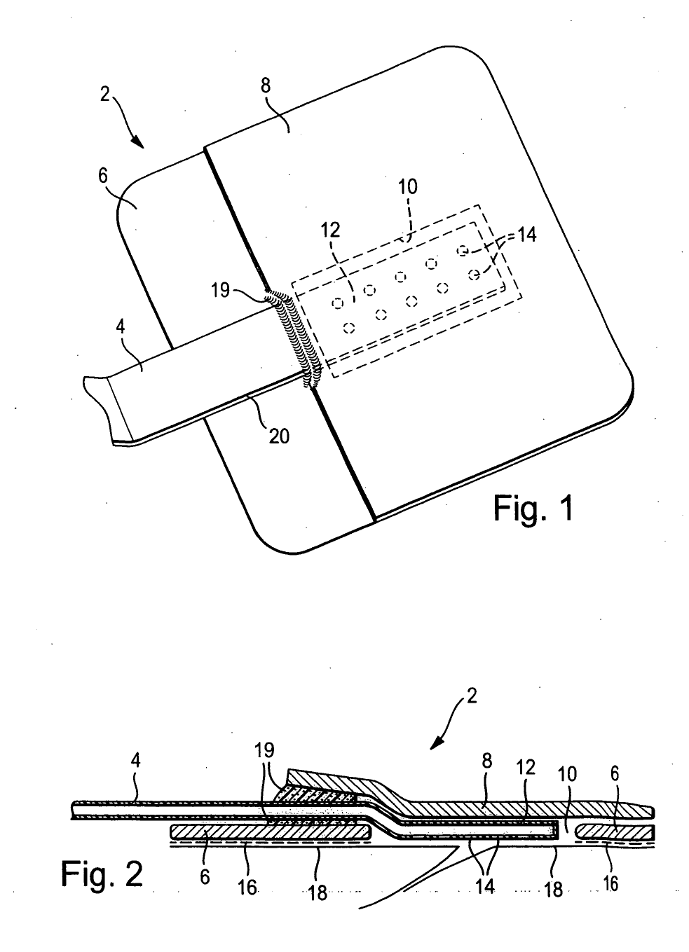

FIG. 2 shows a schematic sectional view of the connecting device according to FIG. 1, not to be taken to scale; and

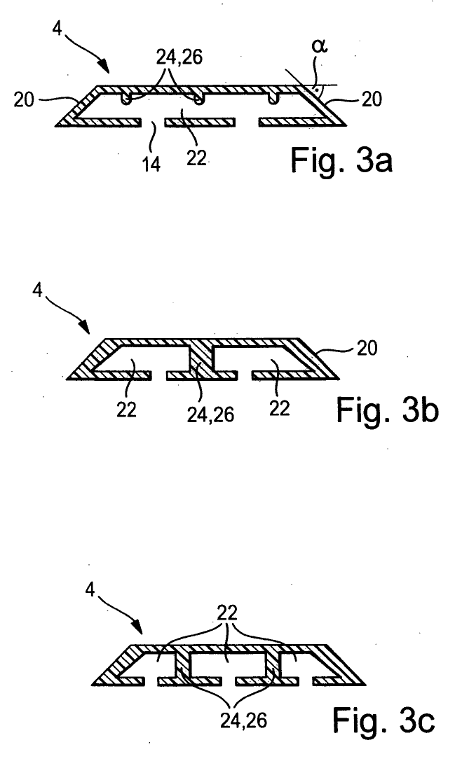

FIGS. 3a to c show sectional views of conduit means of the inventive connecting device (sectional plane perpendicular to the longitudinal extension).

FIGS. 1 and 2 show different views of an inventive connecting device, which is designated in total with reference numeral 2, for use for vacuum treatment of wounds. The illustrated connecting device 2 is disposed, preferably in a detachable fashion, onto an upper side of a vacuum dressing (not shown) facing away from the wound, which extends over a wound to be treated and seals it from the atmosphere in a vacuum-tight fashion.

The connecting device 2 comprises a flat conduit means 4 of an elastomeric flexible material and a flat first carrier means 6 and a flat second carrier means 8 between which the flat conduit means 4 is received like a sandwich, wherein the two flat carrier means 6, 8 hold and support the flat conduit means 4. Pressure or bending forces or torsional forces, which act on the conduit means 4, are thereby uniformly introduced into the composite of the two at least partially overlapping carrier means 6, 8 and are accepted by them.

As is best illustrated in FIG. 2, the first lower carrier means 6 has a broad recess 10, the width of which approximately corresponds to the width of the conduit means 4, such that the conduit means 4 spreads out into this recess 10. A longitudinal section 12 of the conduit means 4 thereby directly faces the vacuum dressing in this area. Several openings 14 are preferably formed in this longitudinal section 12 of the side wall of the conduit means 4 facing the vacuum dressing, which communicate with openings, incisions or slots in the vacuum dressing (not shown).

The lower side of the first carrier means 6 facing the vacuum dressing is either provided with an indicated adhesive coating 16 or is self-adhesive. The overall lower side including the recess 10 is covered by a removable two-part release foil 18 which is removed shortly before application of the connecting device 2 onto the vacuum dressing (not shown).

In the case shown by way of example, the recess 10 in the first carrier means 6 has a rectangular shape such that the conduit means 4 can extend into it in a spread-out and complementary fashion. The second carrier means 8 extends over the first carrier means 6 such that as large as possible a contact surface or a large extensive contact area results between the first and the second carrier means 6, 8 around the recess 10. The upper carrier means 8, however, is offset with respect to the lower carrier means 6 in the outlet area of the conduit means 4. A vacuum-tight laminated connection is formed between the flat conduit means 4 and the two carrier means 6 and 8. A sealing agent 19 is additionally provided in the outlet area of the conduit means 4 between the two carrier means. The sealing agent 19 is e.g. a reactive adhesive, in particular a silicon adhesive.

The two carrier means 6, 8 of this embodiment are formed from hydrocolloid layers, which are preferably self-adhesive, such that the above-mentioned additional adhesive coating 16 is not required. The carrier means may also be made from other materials.

The formation of the conduit means 4 is illustrated in FIGS. 3a to c by means of various embodiments. In a preferred embodiment, the conduit means 4 has a trapezoidal shape and comprises two flanks 20 along its longitudinal extension, which drop at an inclined angle towards the first carrier means 6 and subtend an angle α of approximately 40 to 50° with the plane of extension of the conduit means 4 or the carrier means 6. The conduit means 4 in accordance with FIG. 3a has one single channel 22 which can be loaded with vacuum. In these alternative embodiments according to FIGS. 3b and 3c, two or three channels 22 are formed, wherein the channel of FIG. 3b or the two outer smaller channels according to FIG. 3c may serve as rinsing line for supplying a rinsing medium.

The flat conduit means 4, which is preferably formed on the basis of silicone and is moreover preferably formed in one piece, is provided on its inside with means 24 for preventing collapse of the conduit means 4. These means 24 are formed by ribs 26 which are continuous in the longitudinal direction and are produced in one piece with the conduit means 4. In the embodiment of the conduit means 4 in accordance with FIGS. 3b and 3c, these means 28 are formed by the walls (septums) between the channels 22.

FIG. 2 also indicates the extension in thickness D of the composite of conduit means 4 and carrier means 6, 8. It is advantageously at most 7 mm, in particular at most 5 mm and moreover preferably only 2 to 4 mm.

1-18. (canceled)

19. A connecting device for vacuum treatment of a wound, the connecting device structured to be disposed onto a vacuum dressing which extends over the wound and tightly seals the wound from atmosphere, the device comprising:
a conduit structured to accept loads resulting from evacuation thereof, said conduit having at least one opening in a wall thereof facing the vacuum dressing for communication with a wound space through at least one opening in the vacuum dressing, said conduit being flexible and flat; and
a flat vacuum-tight first carrier, said first carrier holding said conduit in a vacuum-tight fashion, said first carrier having a broad recess with a width which is at least as large as a width of said conduit and a length which is at least 15 mm, wherein said conduit flatly extends into said recess during use to thereby directly face the vacuum dressing via said recess, wherein said at least one opening in said conduit directly communicates with the at least one opening in the vacuum dressing.

20. The connecting device of claim 19, wherein said length of said recess in said first carrier is at least 20 mm, at least 30 mm or at least 40 mm.

21. The connecting device of claim 19, wherein said first carrier has an adhesive coating on a side thereof facing the vacuum dressing, wherein the connecting device is directly applied onto an upper side of the vacuum dressing facing away from the wound in an adhesive and vacuum-tight fashion.

22. The connecting device of claim 19, further comprising a second flat carrier, wherein, in a projection onto a plane of said first carrier, said second carrier completely extends over said recess and said conduit to sandwich and hold said conduit between said first carrier and said second carrier in a vacuum-tight fashion.

23. The connecting device of claim 22, wherein said second carrier is offset with respect to an extension of said first carrier in an area of an outlet of said conduit to form a non-overlapping area of said conduit at that location.

24. The connecting device of claim 22, further comprising a sealing agent for sealing between said first and said second carrier.

25. The connecting device of claim 24, wherein said sealing agent is on a basis of silicon and simultaneously promotes adhesion between said first and said second carriers.

26. The connecting device of claim 19, wherein said first carrier comprises a thermoplastic elastomer of silicon, polyurethane, a hydrocolloid material or a self-adhesive hydrocolloid material.

27. The connecting device of claim 19, wherein said conduit is formed from an elastomeric material having a Shore A-hardness of maximally 60, of 5 to 60, of 10 to 60, of 15 to 50, of 15 to 40 or of 15 to 35.

28. The connecting device of claim 22, wherein a combined thickness of said conduit and at least one of said first and said second carriers is at most 10 mm, at most 7 mm, at most 6 mm or at most 5 mm.

29. The connecting device of claim 19, wherein a width of said conduit is at least 10 mm, at least 15 mm, at least 18 mm, at most 30 mm or at most 25 mm.

30. The connecting device of claim 19, wherein, as viewed in cross-section perpendicularly to a longitudinal extension of said conduit, said conduit has a continuous tubular shape in a peripheral direction thereof.

31. The connecting device of claim 19, wherein said conduit comprises internal structures for preventing collapse of said conduit under vacuum load.

32. The connecting device of claim 31, wherein said structures are integral with a material of said conduit.

33. The connecting device of claim 31, wherein said structures define ribs or projections.

34. The connecting devise of claim 33, wherein said ribs or projections extend continuously in a longitudinal direction of said conduit.

35. The connecting device of claim 19, wherein said conduit has several channels which are separated from each other in a vacuum-tight fashion.

36. The connecting device of claim 35, wherein said conduit is formed in one piece.

37. The connecting device of claim 22, wherein at least one of said first and said second carrier is formed from a flexible elastomeric material of a Shore A-hardness of 5 to 60, 10 to 60, 15 to 50, 15 to 40 or 15 to 35.

38. The connecting device of claim 19, wherein a flat extension of said first carrier including said recess is at least 1.5 times or at most 5 times an area, projected perpendicularly onto said first carrier, of said conduit.

39. The connecting device of claim 19, wherein said conduit has a trapezoidal cross-section.

40. The connecting device of claim 19, wherein a clearance area of said at least one opening in said conduit is 5 to 50% of a surface of said recess.
